(19) United States
(12) Patent Application Publication
(10) Pub. No.: US 2020/0367548 A1
(43) Pub. Date: Nov. 26, 2020

(54) Indigenous and Improved Bacterially Fermented CBD, CBG and Related Cannabinoid Oral Dosage Forms

(71) Applicant: BYO Holdings, LLC, Koshkonong, MO (US)

(72) Inventor: Jordan Seth Rubin, College Grove, TN (US)

(73) Assignee: BYO Holdings, LLC, Koshkonong, MO (US)

(21) Appl. No.: 16/839,343

(22) Filed: Apr. 3, 2020

Related U.S. Application Data
(63) Continuation-in-part of application No. 16/148,462, filed on Oct. 1, 2018.
Application No. 16/148,462 is a continuation of application No. 15/076,931, filed on Mar. 22, 2016, now abandoned.
Application No. 15/076,931 is a continuation-in-part of application No. 16/270,033, filed on Feb. 7, 2019.
Application No. 16/270,033 is a continuation of application No. 15/204,280, filed on Jul. 7, 2016, now abandoned.
(60) Provisional application No. 62/912,930, filed on Oct. 9, 2019.
(60) Provisional application No. 62/138,099, filed on Mar. 25, 2015.
(60) Provisional application No. 62/190,039, filed on Jul. 8, 2015.

(51) Int. Cl. A23P 10/30; C12N 1/20; A61K 35/742; A61K 35/747; A23L 33/135; A23L 2/38; A23L 11/00; A61K 36/00

(52) U.S. Cl. CPC A23P 10/30; C12N 1/20; A61K 35/742; A61K 35/747; A23Y 2220/67; A23L 2/382; A23L 11/09; A61K 36/00; A23L 33/135

(57) ABSTRACT

The present invention is a cannabidiol (CBD), cannabigerol (CBG) or related cannabinoid oral dosage form including bacterially fermented hemp, typically compounded as a tablet or formulated within a capsule or as an additive to other dietary supplements or functional foods. The dosage forms contain dietary fiber, important to activity as the desired delivery system, having a ratio of one part soluble dietary fiber to 30 parts insoluble dietary fiber, which is an optimal fiber ratio for oral delivery and bioavailability of cannabidiol, cannabigerol and other desirable cannabinoids from either activated or native hemp.

### CROSS-REFERENCE TO RELATED APPLICATIONS

This patent application claims priority to U.S. Provisional Patent Application 62/912,930 filed 9 Oct. 2019, and is also a Continuation-in-Part of U.S. Ser. No. 16/148,462 filed 1 Oct. 2018, which is a continuation of U.S. Ser. No. 15/076,931 filed 22 Mar. 2016, which claims the benefit of U.S. Provisional Patent Application No. 62/138,099 filed 25 Mar. 2015, each of which is incorporated herein by reference.

### FIELD OF THE INVENTION

The invention pertains to improved CBD and CBG (and related cannabinoid) delivery methods and dosage forms, namely, bacterially fermented hemp.

### BACKGROUND OF THE INVENTION

Cannabis indica, Cannabis sativa and Cannabis ruderalis are well established both as herbs and pharmaceuticals throughout history. At the same time, bacterial fermentation of comestibles of all kinds, including but by no means limited to Lactobacillus, has a comparably august track record in both cuisine and medicament innovation, and bacterial fermentation is so central to modern well-being that it is easy to lose sight of how ubiquitous comestible bacteria are. Life without cheese, buttermilk, kefir, yogurt, kim chi, sauerkraut, fermented legumes, wine, and beer, just to name a few, would have been as unthinkable, hundreds of years ago, as it is today—and of course all of these things rely on bacterial fermentation for their sheer existence.

While bacterial fermentation in the food and beverages industries has enjoyed a seemingly mature—or at least multiple-centuries-old—implementation in Western culture and economics, the relatively recent legality of hemp growing especially in the United States has led to a predictable avalanche in the cannabis industry. Cannabis extraction, formulation and processing in the United States, with legal avenues thanks to the 2018 Farm Bill, now regularly yield cannabidiol—CBD—and a myriad of products containing it. As a result, more-retail-cash-registers-than-not offer various CBD products for sale—sometimes many edible offerings such as individually wrapped chocolates containing CBD along with CBD topicals, oils and capsules—and of course online sales are brisk. CBD has long been known for its pain relieving, relaxing, sleep supporting, anxiety reducing benefits, rather than intoxicant properties, and sales of CBD containing products have been understandably swift and growing in the U.S. from 2018 to date.

As with any newly popular ingredient, there are opportunities for high quality products as well as those of lesser value and benefit. There are currently reliable, responsible hemp growers and manufacturers and, presumably, also formulators and peddlers reminiscent of the “snake oil salesmen” of the American 1800s. Chinese snake oil was a legitimate anti-inflammatory substance for decades if not centuries, prior to fake iterations that appeared later in the U.S. The original Chinese snake oil was made from the oil of the Chinese water snake, which was rich in the omega-3-fatty acids that are known to reduce inflammation. This “snake oil” in its original form was indeed effective as a topical medicament to treat arthritis and bursitis and, eventually, the putative product made its way to the United States—if not the omega-3-rich water snake itself, or its curative extract. The point here is that with CBD, as with anything else, responsible sourcing, processing and quality control in manufacturing are the bedrock of any superior pharmaceutically active agent. The pressures of manufacturing in light of a population clamoring for CBD are particularly intense, in world in which side-effect- or addiction-minimized pain management is still an elusive if (not scandal-laden) goal.

It is interesting that, as a general practice regarding naturally-occurring active agents—and particularly those of herbal sources—there seems to be a knee-jerk compulsion to extract the active agent compound from its botanical herb or spice. Extracting and synthesizing digitalis from foxglove, a natural herb, is of course a paradigm in pharmaceutical history. In theory there is nothing wrong with extraction processes—although in practice there can indeed be negative implications to extraction, in particular as to the molecule(s) to be extracted. Extraction agents such as petroleum or coal-tar derived solvents can create residues or even alter the chemical composition of the sought-after molecule. Worse, beneficial co-factors present in the natural product, in this case an herb, can be separated from the active agent so as to lose the synergy of administration of the whole herb with its known and yet-to-be discovered compounds. Even today, when Cannabis indica, Cannabis sativa and Cannabis ruderalis are on the brink of becoming “health food” [so to speak] instead of “Just Say No!” fodder, the temptation seems to be ubiquitous to extract and isolate key constituents within them, in order to obtain their active agent(s) for further commercialization. The question which the present inventor asked, therefore, was—whether traditional extraction or isolation is the only processing method that can deliver the true benefits of hemp? And, if extraction or isolation is not the best approach, what additional beneficial conversions are possible to enhance the power and constituency of hemp for nutritional and dietary supplementation?

### SUMMARY OF THE INVENTION

The present supplement, and pharmaceutical, nutraceutical and treatment method and method for delivering an active agent, centers on a powdered form of a bacterially fermented native or activated unextracted hemp containing cannabinoids including but not limited to cannabidiol (CBD) and cannabigerol (CBG), dietary fiber of a particular ratio, vitamins, minerals, flavonoids, terpenes, fatty acids and amino acids. The powder can be optionally blended with other botanical ingredients and compressed into a tablet form for administration to or consumption by an animal or human in need of a reliably sourced CBD (or other cannabinoid) oral dosage form. Fermentation is conducted with any bacterium suitable for consumption or oral administration, including but not limited to Bacillus subtilis, Bacillus subtilis ssp Natto, Bacillus coagulans, Lactobacillus plantarum. Acetobacter pasteurianus, Acetobacter ghanensis, Komagataeibacter oboediens, or Komagataeibacter saccharivorans, used alone or in combination in any given culture. Such a dosage form containing the inventive powders retains live bacteria from the fermentation, as a probiotic fraction of the dosage form, as well as a retained minor fraction of water (moisture). While the present bacterially fermented native or activated hemp is well suited for use alone, it may be admixed with other ingredients, whether active agents or excipients, fillers or comestible ingredients comprising dietary supplements or functional food ingredients. Hemp for the purpose of this patent application is hemp which meets the standards of the 2018 Farm Bill, that is, is a native Cannabis (various species and varieties) which contains no more than 0.3% tetrahydro cannabinol (THC) on a dry weight basis. Activated hemp is hemp that has been heated or treated, with more discussion regarding activation and its consequences' appearing later in this specification. Native hemp is raw, that is, unheated and untreated, hemp. Native or activated hemp which remains unextracted, for the purposes of the present bacterial fermentation and further steps, contains interesting naturally occurring co-factors, known and currently unknown, including without limitation other cannabinoids, dietary fiber, fatty acids, amino acids, terpenes and flavonoids. These known and unknown co-factors inevitably enhance any or all of delivery, bioavailability and efficacy of the CBD/cannabinoid(s) in vivo. This invention takes the native or activated hemp starting material and subjects it to bacterial fermentation, both for the creation of a probiotic profile in the resulting end product and also beneficially to convert and enhance the constituent molecules and compounds in the hemp to an increasingly diverse and prized spectrum of nutrients and active agents—all while keeping the hemp in its unextracted state. A key part of the present invention therefore inheres in the synergy achieved by using unextracted hemp as the starting material for a bacterial fermentation, and the deploying the fermentation product as a further constituent, usually powdered, in a tablet or capsule (or equivalent) ingredient as a CBD/CBG/cannabinoid oral dosage form or in other comestible dietary supplements or functional foods. Careful drying techniques are used after fermentation, especially for “native” versus “activated” hemp, which do not denature the hemp constituent fraction or harm the bacteria, usually at low heat and controlled moisture. Another key component to the invention is in the engineering of the delivery system, with a beneficial soluble dietary fiber (SDF)/insoluble dietary fiber (IDF) ratio, in the unextracted native or activated hemp itself, of 1:30 SDF/IDF, allowing for effective formulation and delivery of key constituents. When native or activated hemp are bacterially fermented, the action of the bacteria increase at least the cannabidiol (CBD) and cannabidiol acid (CBDA) constituency, enhancing the presence of the most desirable cannabinoids but NOT the THC.

This combined increase in CBD plus CBDA, or the total of the two, is between about 12% to 300%, compared to the starting constituency of the hemp prior to fermentation and as to the hemp portion of the fermentation product itself.

### DETAILED DESCRIPTION OF THE INVENTION

As with popular or over-the-counter dosage forms, particularly for active agents known to control pain, dosing is tantamount to safe and effective treatment. When it comes to CBD or CBG, general dosing guidelines suggest that a recommended initial dose is somewhere in the range of 1-5 mg, once or twice a day, for an averaged sized human patient, with possible ethical dosing of up to 20 mg or more taken as often as three times per day. Veterinary dosing is generally pro rata by body mass/weight at similar levels. As the CBD industry matures, inevitably further dosing guidelines will become available—but as with all active agents a serious challenge is to prevent inadvertent (or intentional) overdose. One benefit of the present formulations inheres in the retained diluent of the whole native hemp or activated hemp, compared to other dosage forms that contain extracts, which retained diluent in turn assures relatively lower dosing in the inventive material per se. In addition to the lower cannabinoid (i.e. CBD, CBG) concentration, along with synergistic co-factors which provides a more balanced, “whole food” effect with less chance of deleterious side effects, the ability of the present bacterial fermentation to increase the profile of desirable cannabinoids only enhances the value of the present hemp product as a dietary supplement and wellness aid. In addition, the ratio of soluble dietary fiber (SDF) to insoluble dietary fiber (IDF) remains the same as in the starting hemp. This SDF/IDF ratio is 1:30, that is, for every 1 part of soluble dietary fiber in the hemp (native or activated), there is also 30 parts insoluble dietary fiber. This SDF/IDF ratio is discussed further, below.

If native or activated hemp has, for example, a starting content of 10% CBD, one gram of the hemp starting material will contain about 100 mg CBD. When one cultures one gram hemp with bacteria, no matter how the bacterial culture proceeds the bacteria—even when still viable—add negligible weight to the finished fermented product. In other words, in the fermented hemp end product after careful drying, the presence of the dried probiotic bacteria constitute only 1% or less of the mass of the fermented hemp. Accordingly, 1 g dry weight of hemp, after bacterial fermentation, continues to contain 10% CBD if its starting content was 100% CBD and, after factoring in a 12% to 300% increase in CBD/CBDA or both due to the bacterial action, the cannabinoid dose per gram transitions to 112 mg to 300 mg per gram of the final fermented hemp product. It is therefore easily possible for those skilled in the art to measure, titrate and check products according to the invention to include the desired amount of bacterially fermented hemp into a unit dosage form, to provide unit dosage forms that contain, for example, 1 mg, 3 gm, 5 mg, 10 mg and even 20 mg CBD dosage forms by selecting the appropriate amount of the dried fermented material. Larger serving sizes of tablets or capsules can thus contain up to 20 mg CBD while still having a size that can be easily consumed without difficulty. The ability of hemp with bacterial fermentation to be formulated into oral dosage form capsules—or to be tabletted directly without additives—means that creation of dosing per unit is straightforward and well within the skill of the art. The combination itself, of taking hemp—native or activated—and fermenting it with bacteria prior to creating one or more dosage forms from the fermentation product, is the gravamen of the present invention.

As set forth above, the present hemp starting material is fermented with any suitable bacterium, including but not limited to Bacillus subtilis, Bacillus subtilis ssp Natto, Bacillus coagulans, Lactobacillus plantarum. Acetobacter pasteurianus, Acetobacter ghanensis, Komagataeibacter oboediens, or Komagataeibacter saccharivorans, The presence of one or more of these probiotics and their fermentation products (and viable cultures) is the main exception, besides moisture, to the premise that, overall, the dosage form material is predominantly or completely “whole” hemp. In other words, except for further formulations in functional foods and other products in which the present fermented product can be an ingredient, it is important to recognize the versatility—and even the conformability for tabletting and the like—of just the bacterially fermented hemp that is subsequently gently dried and comminuted—without need for a lot more processing or formulation as so many other active agents typically do need.

It almost goes without saying that organically sourced hemp, extracted without hydrocarbon-based or petroleum or coal tar derived solvents, is the best choice for the hemp starting material according to the present invention. By using organic hemp and avoiding noxious extraction solvents, the presence of pesticides or other solvent residues or undesirable adulterants in the hemp is reduced to a beneficial minimum. Not only is the reduction of these extraneous contaminants good in and of itself, but the absence of unwanted residues maximizes the original confluence of the indigenous cannabinoids such as CBD with its synergistic co-factors, known (see list above) or unknown.

Bacterially fermented hemp according to the present invention is typically dried, possibly further “activated” through decarboxylation and co-minuted prior to tabletting or encapsulating. Dehydration to a moisture content of below 15%, preferably below 10% and more preferably to 5-6% is important in the creation of the present oral dosage forms. The co-minution may be but need not be to a (small) particle size generally within the range of powders. Generally speaking, bacterially fermented hemp particles of at least 100 microns in diameter, up to irregularly shaped particles of up to about 5 mm in their longest dimension, are best for tabletting or encapsulating according to the present invention. Surprisingly, hemp/probiotic particles of this size are beneficially self tabletting without added ingredients and with a minimum of compression energy, that is, not enough pressure to generate significant heat. Avoidance of excessive processing also prevents the generation of unwanted heat that can denature cannabinoids (CBD), terpenes or additional cofactors in the hemp. Having said that, however, the administration of hemp/bacterial probiotic as a powder (that is, in traditional powder particle size distributions smaller than 100 microns) and as predominantly the only oral dosage form constituent as described above—is still within the scope of the present invention.

The primary disclosure of this patent application is directed to dosage forms in which—with few exceptions such as added inert excipients, probiotics, botanicals, vitamins and minerals or adjusted or retained moisture—bacterially fermented hemp is the main ingredient in an oral dosage form. Having said that, the subject bacterially fermented hemp has all sorts of secondary benefits as additives to other dietary supplements, nutritional dosage forms and functional foods—virtually without limitation as to any application where probiotics can be added.

As disclosed above, hemp (prior to bacterial fermentation) contains total dietary fiber (TDF) having a ratio of 1 part SDF to 30 parts IDF. As compared to higher SDF-containing botanicals, such as for example oat bran or bananas, a ratio of 1:30 SDF/IDF is a notably low SDF/IDF ratio and, for the purposes of the present invention, this high inclusion of IDF is extremely beneficial to delivery of CBD and other cannabinoids from an oral dosage form. SDF, upon oral administration, tends to create a sol/gel in the gastrointestinal tract, which in turns tends to retain in solution, i.e. binding or suspension, other molecules in its vicinity such as, in this case, cannabinoids. In other contexts, SDF is a highly desirable nutrient, that can even be partially digested by bacteria in the gut, but in the context of a cannabinoid delivery system SDF actually creates a binding system and subsequent removal from the body for an active agent, rather than a true delivery (release) system into the blood stream. By contrast, the high IDF inclusion assures the desirable release of the active agent promptly if not instantly in the stomach or upper gastrointestinal tract. Given this understanding of how the present oral dosage form works, moreover, it may be seen that the present oral composition, although botanical in initial source, is a highly engineered composition and not merely a product of nature at all. With the present oral dosage forms, the cannabinoid content is reduced (compared to native hemp) and yet the SDF/IDF ratio of 1:30, typical of native hemp, enhances delivery due to its high soluble fiber fraction.

Important cannabinoids in hemp are not limited to cannabidiol (CBD). Known significant cannabinoids other than THC include, without limitation, cannabigerol (CBG), cannabidivarin (CBDV), cannabichromene (CBC), cannabinol (CBN) and combinations thereof. Various strains of hemp tend to present different ratios of these cannabinoids and, in due course, the desired ratios will also inevitably be genetically engineered if not traditionally cross-bred. The ability of whole, unextracted (native or activated) hemp that is bacterially fermented is thus able to serve as a uniquely effective delivery system for any and all cannabinoids and any additional beneficial hemp components present, as to any hemp strain now known or developed in the future.

A number of interesting and beneficial things happen to hemp when it is subjected to bacterial fermentation. Unlike with fungal fermentation of spent extracted hemp, in which the hemp inevitably has to be sterilized prior to fungiculture, bacterial fermentation provides its own de facto sterilization (by competitive overgrowth dynamics) so that constituents of the hemp that are particularly desirable are not denatured due to excessive heating, when native hemp is the starting material. An example is terpenes—which are plentiful in non-heat-treated hemp and do not disappear before or during bacterial fermentation. Therefore, bacterial fermentation can be applied to either “activated” hemp that has gone through a decarboxylated process, typically heat and pressure, or a native hemp that is raw and unheated. When desired, activated and raw/native hemp may be admixed for bacterial fermentation. The action of bacteria also creates metabolites with the substrate such as organic acids (acetic, malic, formic . . . ) which contribute to nutritive and active agent profiles and constituent diversity. Perhaps most important of all, fermentation of hemp with bacterial culture both increases and stabilizes (see Example 2 below) the cannabinoid acids (CBDA, CBGA etc) in the hemp, which is important in preserving the anti-nausea, anti-withdrawal symptom and pain management aspect of cannabinoid acids (un-de-carboxylated) themselves. Of course having all these benefits together with a novel probiotic makes for a much needed, new and useful composition, for administration in unit dosage form.

### Example 1

As a test to illustrate the fiber ratios in native or activated hemp, a quantity of native hemp was subjected to a traditional extraction of cannabinoids (not a part of the invention herein) by moderate crushing and extraction of cannabinoids to create a “hemp pomace” which continued to include cannabinoids therein. The extraction was performed by carbon dioxide solvent extraction. The resulting pomace was carefully air dried at 115 degrees Fahrenheit to prevent denaturing of all compounds and compositions in the pomace. A representative dried pomace prepared according to the above method steps contained 6% moisture and certain exemplary specifications listed in the below table. Because only liquid was extracted from the native hemp, the fiber ratios of the resulting dried pomace were representative of the fiber ratios in the starting native hemp (one form of hemp used in the bacterial fermentation of the present invention). The fiber ratios of insoluble dietary fiber (IDF) to soluble dietary fiber (SDF), not shown in the below table, were 1:30 SDF/IDF.

TABLE I

| QD252 - Protein - Combustion | Reference | Accreditation | Analysis Completed |
|---|---|---|---|
| | AOAC 990.03; AOAC | A2LA ISO/IEC | Jan. 15, 2020 |
| | 992.15 | 17025: 2005 | |
| Parameter | Result | | |
| Protein | 26.50% | | |
| Nitrogen - Combustion | 4.24% | | |
| Protein Factor | 6.25 | | |
| QD250 - Ash | Reference | Accreditation | Analysis Completed |
| | AOAC 942.05 | A2LA ISO/IEC | Jan. 15, 2020 |
| | | 17025: 2005 | |
| Parameter | Result | | |
| Ash | 17.74% | | |
| QD226 - Calories, Calculated | Reference | Accreditation | Analysis Completed |
| | CFR - Atwater | A2LA ISO/IEC | Jan. 17, 2020 |
| | calculation | 17025: 2005 | |
| Parameter | Result | | |
| Calories Calculated | 323 kcal/100 g | | |
| QD038 - Carbohydrates, | Reference | Accreditation | Analysis Completed |
| | CFR 21-calc. | A2LA ISO/IEC | Jan. 17, 2020 |
| | | 17025: 2005 | |
| Parameter | Result | | |
| Carbohydrates, Calculated | 46.25% | | |
| QD148 - Moisture by Vacuum | Reference | Accreditation | Analysis Completed |
| | AOAC 925.09 | A2LA ISO/IEC | Jan. 17, 2020 |
| | | 17025: 2005 | |
| Parameter | Result | | |
| Moisture and Volatiles - Vacuum | 6.0% | | |
| Oven | | | |
| QD251 - Calcium by ICP | Reference | Accreditation | Analysis Completed |
| | AOAC 984.27 | A2LA ISO/IEC | Jan. 15, 2020 |
| | mod, 927.02 | 17025: 2005 | |
| | mod | | |

### Example 2

Six samples of an activated hemp composition, all of the same mass, were individually autoclaved at 170 degrees F. for four hours, 170 degrees F. for two hours, 200 degrees F. for four hours, 200 degrees F. for two hours, 250 degrees F. for four hours and 250 degrees F. for two hours. The hemp starting material contained 75% native hemp and 25% of a noncannabinoid spice mix that was uniform throughout the tests conducted and reported in this example. After the autoclaving step, expected to accomplish at least partial decarboxylation of organic acids in the hemp material including cannabinoid acids, the cannabidiol, cannabidiol acid and total constituency of the autoclaved hemp material was measured and is shown in the following table as to the six described samples.

TABLE II

| Sample No. | CBD % | CBDA % | Total CBD + CBDA % |
|---|---|---|---|
| 1 | 1.95 | 0.73 | 2.60% |
| 2 | 3.1 | 1.01 | 4 |
| 3 | 5.4 | 0.19 | 5.6 |
| 4 | 5.4 | 0.47 | 4.8 |
| 5 | 5 | non-detectable | 5 |
| 6 | 6.1 | non-detectable | 6.1 |

Samples identical to 1-6 after autoclaving were relabelled Samples 7-12 and were inoculated and incubated with Acetobacter, under identical inoculation and incubation parameters for all samples. After incubation, the CBD, CBDA and total combined CBD+CBDA constituencies were measured and reported as shown in the following table.

TABLE III

| Sample No. | CBD % | CBDA % | Total CBD + CBDA % |
|---|---|---|---|
| 7 | 6.3 | 3.2 | 9.10% |
| 8 | 2.2 | 2.4 | 4.4 |
| 9 | 6.1 | 0.32 | 6.3 |
| 10 | 6.2 | 0.5 | 6.6 |
| 11 | 7.1 | non-detectable | 7.1 |
| 12 | 9.3 | non-detectable | 9.3 |

The above data (eliminating the outliers) illustrate that it is possible to increase individual CBD or CBDA constituents, or the combination of the two, by about a twelve percent increase up to about a 300% increase simply by the bacterial fermentation of the cannabinoid starting material.

Although the technology has been described with particularity above, with reference to specific materials and methods, the invention is only to be limited as is set forth in the following claims.

I claim:

1. A cannabinoid oral dosage form in unit dosage form, consisting essentially of bacterially fermented hemp, compounded as a tablet or within a capsule, powder or other unit dosage form, having a soluble dietary fiber to insoluble dietary fiber ratio of 1:30, having a maximum cannabinoid dose per unit dosage form of 25 mg, and having viable bacteria therein as a probiotic dietary supplement.

2. The cannabinoid oral dosage form according to claim 1, wherein said bacterially fermented hemp contains cannabinoid acids in chemically stabilized form.

3. A cannabinoid oral dosage form in unit dosage form, consisting essentially of bacterially fermented hemp, compounded as a tablet or within a capsule, powder or other unit dosage form as the predominant or exclusive ingredient and having increased cannabinoid in the amount of 12-300 percent compared to the starting hemp material prior to bacterial fermentation.

4. The oral dosage form of claim 1, wherein each oral dosage form contains less than 10% water as moisture and wherein said cannabinoid is one or more of cannabidiol, cannabigerol, cannabidivarin, cannabichromene, or cannabinol.

5. The oral dosage form of claim 1, wherein said hemp is fermented with one or more bacteria selected from the group consisting of Bacillus subtilis, Bacillus subtilis ssp Natto, Bacillus coagulans, Lactobacillus plantarum, Acetobacter pasteurianus, Acetobacter ghanensis, Komagataeibacter oboediens, and Komagataeibacter saccharivorans prior to compounding.

6. The oral dosage form of claim 1, wherein said dosage form contains no more than 10 mg cannabinoid per unit dosage form.
